(19) United States
(12) Patent Application Publication
(10) Pub. No.: US 2016/0130336 A1
(43) Pub. Date: May 12, 2016

(54) ANTI-VEGF ANTIBODIES AND USE THEREOF

(71) Applicants: DEVELOPMENT CENTER FOR BIOTECHNOLOGY, New Taipei City (TW); DCB-USA LLC, Wilmington, New Castle, DE (US)

(72) Inventors: Jiann-Shiun Lai, New Taipei City (TW); Yan-Da Lai, New Taipei City (TW); Yen-Yu Wu, New Taipei City (TW); Yi-Jiue Tsai, New Taipei City (TW); Yu-Ying Lin, New Taipei City (TW)

(73) Assignees: Development Center for Biotechnology, New Taipei City (TW); DCB-USA LLC, Wilmington, New Castle, DE (US)

(21) Appl. No.: 14/895,297

(22) PCT Filed: Dec. 29, 2014
(86) PCT No.: PCT/US2014/072557

§ 371 Date: Dec. 2, 2015

Related U.S. Application Data
(60) Provisional application No. 61/922,537, filed on Dec. 31, 2013.

(51) Int. Cl. C07K 16/22

(52) U.S. Cl. CPC C07K 16/22; C07K 2317/565; C07K 2317/55; C07K 2317/622; A61K 2039/505

(57) ABSTRACT

An anti-VEGF antibody, or a binding fragment thereof, includes a heavy-chain variable region that comprises: (1) a CDRH1 sequence selected from SEQ ID NO: 17, 20, 23, 26, 29, 32, 35, or 38), (2) a CDRH2 sequence selected from SEQ ID NO:18, 21, 24, 27, 30, 33, 36, or 39, and (3) a CDRH3 sequence selected from SEQ ID NO:19, 22, 25, 28, 31, 34, 37, or 40; and a light-chain variable region that comprises: (1) a CDRL1 sequence selected from SEQ ID NO: 41, 44, 47, 50, 53, 56, 59, or 62, (2) a CDRL2 sequence selected from SEQ ID NO: 42, 45, 48, 51, 54, 57, 60, or 63, and (3) a CDRL3 sequence selected from SEQ ID NO: 43, 46, 49, 52, 55, 58, 61, or 64. A method for treating or preventing a VEGF-related disorder, e.g., diabetic retinopathy, age-related macular degeneration, or cancer, uses the antibodies.

### FIELD OF THE INVENTION

This invention relates to VEGF antibodies, particularly to the generation and uses of such antibodies.

### BACKGROUND OF THE INVENTION

Vascular endothelial growth factor (VEGF) is a signal protein that can stimulate vasculogenesis (a process of creating blood vessels during embryonic development) and angiogenesis (a process of forming new blood vessels from existing blood vessels). Due to its angiogenic property, VEGF can restore the oxygen supply to tissues when blood circulation is inadequate, e.g., after injury.

When VEGF in the tissue is overexpressed, it can result in disease conditions. For example, overexpression of VEGF leads to vascular disease in the retina (e.g., diabetic retinopathy). In addition, solid tumors cannot grow beyond a limited size without an adequate blood supply to provide the necessary nutrients for the growth. To overcome this limitation, solid tumors express VEGF that enables them to grow and metastasize.

VEGF family members include VEGF-A, VEGF-B, VEGF-C, VEGF-D, and PGF (placenta growth factor). Among these, VEGF-A is the most import factor and regulates normal and pathological angiogenesis (i.e., blood vessel formation). VEGF-C and VEGF-D regulate lymphatic vessel formation. VEGF-A includes several protein isoforms—VEGF₁₂₁, VEGF₁₆₅, VEGF₁₈₉, and VEGF₂₀₆ (the subscripts indicate the lengths of the proteins), which can regulate physiological functions via different physiological characteristics and extracellular proteolysis. For example, VEGF₁₂₁ is the only VEGF that cannot associate with heparin and therefore cannot adhere to cell surfaces. VEGF₁₆₅ has exocrine properties and can adhere to extracellular matrix. Among these, VEGF₁₈₉ is a protein with many basic amino acids (high pI value) and is almost completely adsorbed on cell surfaces. VEGF₁₆₅ is the major isoform with the physiological activity. VEGF degraded by extracellular proteases affects its bioavailability. For example, plasmin can cleave VEGF₁₆₅ or VEGF₁₈₉ to release a fragment containing the first 110 residues that retains the bioactivity. However, plasmin can also digest VEGF fragments to reduce its mitotic activity.

Blood vessel formation in normal tissues is regulated by VEGF. In this process, several differentiated epithelial cells play different roles. For example, tip cells may form stalk cells, which then proliferate over the tip cells to form blood vessel lumen. Under normal conditions, vascular endothelial growth factor (VEGF) and anti VEGF exist in balance. Therefore, blood vessels can form and differentiate normally. In the process of tumorous growth, this balance is lost and a network of blood vessel forms around the tumor to provide the blood and nutrients needed by the tumor cells, thereby the tumor cells can continue to grow and metastasize.

Since the 1970s, the physiological functions of VEGF have been gradually elucidated. Pharmaceuticals have been developed based on the theory that inhibition of angiogenesis can inhibit and cure cancer. Some of these pharmaceuticals have emerged on the market. Currently, the main anti-angiogenesis antibody pharmaceutical is Avastin® (bevacizumab). In 2011, the total sales of Avastin® in the U.S. reached 6.193 billion dollars (Med. Ad. News, October 2011). Avastin® is effective in treating lung cancer, breast cancer, colon cancer, kidney cancer, brain cancer, and other malignant tumors. In addition to cause blood vessel shrinkage around tumors, Avastin® also helps other chemotherapeutic agents to infiltrate the tumor to exert their effects.

Because anti-VEGF reagents can be useful pharmaceuticals, there is still a need for antibodies that can be used to treat or control VEGF-related disorders.

### SUMMARY OF THE INVENTION

Embodiments of the invention relate to antibodies that can inhibit VEGF functions. The antibodies may be polyclonal or monoclonal antibodies.

In one aspect, the invention relates to anti-VEGF antibodies capable of neutralizing an activity of VEGF. Antibodies in accordance with one embodiment of the invention may be polyclonal or monoclonal antibodies. In accordance with embodiments of the invention, antibodies can prevent the disorders associated with undesired activities of VEGF, such as cancer growth and metastasis.

In accordance with embodiments of the invention, an antibody or a binding fragment thereof (e.g., an scFv, Fab or F(ab)₂ fragment thereof), comprises a heavy chain variable region (V_{H}) that comprises CDRH1, CDRH2, and CDRH3 sequences, wherein the CDRH1 sequence is selected from SEQ ID NO: 17, 20, 23, 26, 29, 32, 35, or 38, wherein the CDRH2 sequence is selected from SEQ ID NO: 18, 21, 24, 27, 30, 33, 36, or 39, and wherein the CDRH3 sequence is selected from SEQ ID NO: 19, 22, 25, 28, 31, 34, 37, or 40.

In accordance with some embodiments of the invention, an antibody or a binding fragment thereof (e.g., an scFv, Fab or F(ab)₂ fragment thereof), comprises a light chain variable region (V_{L}) that comprises CDRL1, CDRL2, and CDRL3 sequences, wherein the CDRL1 sequence is selected from SEQ ID NO: 41, 44, 47, 50, 53, 56, 59, or 62, wherein the CDRL2 sequence is selected from SEQ ID NO: 42, 45, 48, 51, 54, 57, 60, or 63, and wherein the CDRL3 sequence is selected from SEQ ID NO: 43, 46, 49, 52, 55, 58, 61, or 64.

In accordance with some embodiments of the invention, an antibody or a binding fragment thereof (e.g., an scFv, Fab or F(ab)₂ fragment thereof), comprises a heavy chain variable region (V_{H}) that comprises CDRH1, CDRH2, and CDRH3 sequences, wherein the CDRH1 sequence is selected from SEQ ID NO: 17, 20, 23, 26, 29, 32, 35, or 38, wherein the CDRH2 sequence is selected from SEQ ID NO: 18, 21, 24, 27, 30, 33, 36, or 39, and wherein the CDRH3 sequence is selected from SEQ ID NO: 19, 22, 25, 28, 31, 34, 37, or 40; and comprises a light chain variable region (V_{L}) that comprises CDRL1, CDRL2, and CDRL3 sequences, wherein the CDRL1 sequence is selected from SEQ ID NO: 41, 44, 47, 50, 53, 56, 59, or 62, wherein the CDRL2 sequence is selected from SEQ ID NO: 42, 45, 48, 51, 54, 57, 60, or 63, and wherein the CDRL3 sequence is selected from SEQ ID NO: 43, 46, 49, 52, 55, 58, 61, or 64.

In accordance with any of the above embodiments of the invention, an antibody or a binding fragment thereof (e.g., an scFv, Fab or F(ab)₂ fragment thereof), may bind to one or more epitopes on VEGF having the sequences of SEQ ID NO: 66, SEQ ID NO: 67, and/or SEQ ID NO: 68.

In another aspect, the invention relates to methods for treating or preventing a VEGF-related disorder, such as diabetic retinopathy or cancers (cancer growth or metastasis) by administering to a subject in need thereof an anti-VEGF antibody capable of neutralizing an activity of VEGF.

In accordance with any embodiment set forth above, an antibody may be a monoclonal antibody. In accordance with any embodiment set forth above, an antibody may be a humanized antibody or a complete human antibody.

Other aspects and advantages of the invention will be apparent from the following description and the appended claims.

### BRIEF DESCRIPTION OF THE DRAWINGS

FIG. 1 shows a schematic for generating anti-VEGF antibodies using phage libraries in accordance with embodiments of the invention.

FIG. 2 shows a schematic illustrating an exemplary method for generating anti-VEGF antibodies using phage libraries in accordance with embodiments of the invention.

FIG. 3A shows heavy chain variable region sequences of antibodies of the invention. FIG. 3B shows light chain variable region sequences of antibodies of the invention

FIG. 4A shows CDRH1, CDRH2, and CDRH3 of heavy chain variable region sequences of anti-VEGF antibodies in accordance with embodiments of the invention, as compared with that of Avastin®.

FIG. 4B shows CDRL1, CDRL2, and CDRL3 of light chain variable region sequences of anti-VEGF antibodies in accordance with embodiments of the invention, as compared with that of Avastin®.

FIG. 5 shows a schematic illustrating various assays for the characterization of a full-length antibody of the invention.

FIG. 6A shows a three dimensional structure of VEGF and FIG. 6B shows the binding epitope regions by alanine scanning in accordance with embodiments of the invention.

FIG. 6C shows VEGF binding in ELISA of mutative VEGF by alanine scanning to determine binding epitope regions in accordance with embodiments of the invention,

FIG. 7 shows schematic illustrating a setup for assaying effects on VEGF-induced HUVEC migration by various anti-VEGF antibodies of the invention.

FIG. 8 shows results of various analysis and assays of various anti-VEGF antibodies of the invention.

FIG. 9 shows a flowchart for in vivo assay of inhibition of VEGF-induced tumor growth by anti-VEGF antibodies of the invention.

FIG. 10A shows results of inhibition of tumor growth in vivo by various anti-VEGF antibodies. FIG. 10B shows the average results for each antibody.

### DEFINITIONS

As used herein, the term “antibody” broadly refers to an immunoglobulin molecule or immunologically active portion thereof, i.e., an antigen-binding portion or a fragment thereof. Thus, an antibody comprises at least one (preferably two) heavy (H) chain variable regions (V_{H}), and at least one (preferably two) light (L) chain variable regions (V_{L}). The V_{H} and V_{L} regions can be further subdivided into regions of hypervariability, i.e., the “complementarity determining regions” (“CDR”), interspersed with regions that are more conserved, i.e., “framework regions” (“FR”). Each V_{H} and V_{L} is composed of three CDR's and four FR's, arranged from amino-terminus to carboxy-terminus in the following order: FR1, CDR1, FR2, CDR2, FR3, CDR3, FR4. (see, Kabat et al. (1991) Sequences of Proteins of Immunological Interest, Fifth Edition, U.S. Department of Health and Human Services, NIH Publication No. 91-3242; and Chothia et al. (1987) J. Mol. Biol. 196:901-917, which are incorporated herein by reference).

As used herein, CDRH1, CDRH2, and CDRH3 (or HCDR1, HCDR2, and HCDR3) refer, respectively, to the three complementary determining regions (CDR) in the heavy-chain variable (V_{H}) region, and CDRL1, CDRL2, and CDRL3 (or LCDR1, LCDR2, and LCDR3) refer, respectively, to the three complementary determining regions (CDR) in the light-chain variable (V_{L}) region.

An antibody may include one or more constant regions from a heavy or light chain constant region. The heavy chain constant regions comprise three domains, C_{H1}, C_{H2} and C_{H3}, and the light chain constant region comprises one domain, C_{L}. The variable region of the heavy and/or light chains contain a binding domain that interacts with an antigen, while the constant regions of the antibodies typically mediate the binding of the antibody to host tissues or factors, including various cells of the immune system (e.g., effector cells) and the first component (C1q) of the classical complement system.

As used herein, the term “immunoglobulin” refers to a protein consisting of one or more polypeptides substantially encoded by immunoglobulin genes. Human immunoglobulin genes include the kappa, lambda, alpha (IgA1 and IgA2), gamma (IgG1, IgG2, IgG3, IgG4), delta, epsilon and mu constant region genes, as well as the myriad immunoglobulin variable region genes. Full-length immunoglobulin “light chains” (about 25 KDa or 214 amino acids) are encoded by a variable region gene at the NH2-terminus (about 110 amino acids) and a kappa or lambda constant region gene at the COOH-terminus. Full-length immunoglobulin “heavy chains” (about 50 KDa or 446 amino acids), are similarly encoded by a variable region gene (about 116 amino acids) and one of the aforementioned constant region genes, e.g., gamma (encoding about 330 amino acids).

The term “antigen-binding fragment” of an antibody (or “antibody portion,” or “fragment”) or a “binding fragment of an antibody” refers to a fragment of a full-length antibody, wherein the fragment retains the ability to bind specifically to an antigen. Examples of antigen-binding fragments of an antibody include, but are not limited to: (i) an Fab fragment, which is a monovalent fragment consisting of the V_{L}, V_{H}, C_{L} and C_{H1} domains; (ii) an F(ab′)₂ fragment, which is a bivalent fragment comprising two Fab fragments linked by a disulfide bridge at the hinge region; (iii) an Fd fragment consisting of the V_{H} and C_{H1} domains; (iv) an Fv fragment consisting of the V_{L} and V_{H} domains of a single arm of an antibody, (v) a dAb fragment (Ward et al., (1989) Nature 341:544-546), which consists of V_{H} domain; and (vi) an isolated complementarity determining region (CDR).

Furthermore, although the two domains of the Fv fragment, V_{L} and V_{H}, are coded for by separate genes, they can be joined, using recombinant methods, by a synthetic linker that enables them to be made as a single protein chain, in which the V_{L} and V_{H} regions pair to form a monovalent molecule (known as single chain Fv (scFv); see e.g., Bird et al. (1988) Science 242:423-426; and Huston et al. (1988) Proc. Natl. Acad. Sci. USA 85:5879-5883). Such single chain antibodies are also encompassed within the term “antigen-binding fragment” of an antibody. These antibody fragments can be obtained using conventional techniques known to those skilled in the art, and the fragments are screened for utility in the same manner as for intact antibodies.

### DETAILED DESCRIPTION OF THE INVENTION

Embodiments of the present invention relate to anti-VEGF antibodies and methods of using these antibodies. The uses may include treatments, prevention, or diagnosis of diseases associated with VEGF, such as cancer. Antibodies of the invention may include any suitable antibodies, such as polyclonal antibodies or monoclonal antibodies of all classes, human antibodies, and humanized antibodies made by genetic engineering.

In accordance with embodiments of the invention, anti-VEGF antibodies may be produced using phage display techniques. Phage display and combinatorial methods for generating antibodies are known in the art (see e.g., Ladner et al. U.S. Pat. No. 5,223,409; Fuchs et al. (1991) Bio/Technology 9:1370-1372; Hay et al. (1992), Hum. Antibody Hybridomas 3:81-85; Huse et al. (1989), Science 246:1275-1281).

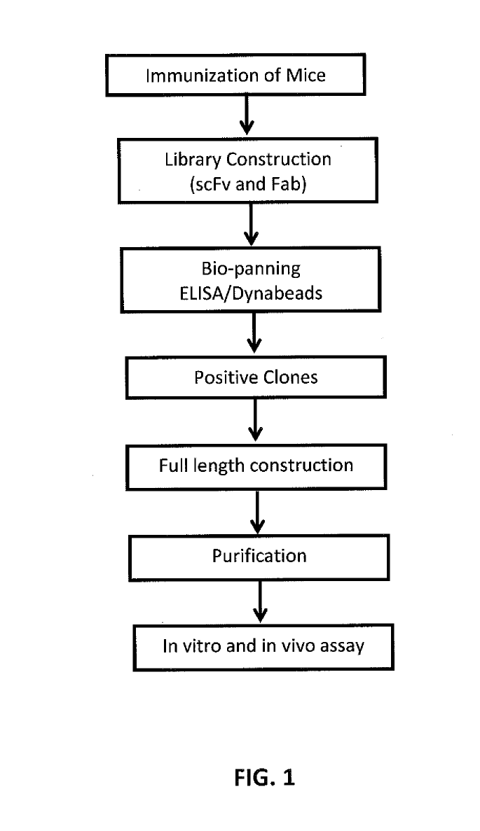

FIG. 1 outlines a general strategy for the production of anti-VEGF antibodies using the phage display approach. With the phage display approach, Fab or scFv are typically produced instead of a whole antibody. However, whole antibodies may be produced by incorporating these sequences into a whole antibody framework. With the phage display approach, mice may be immunized with an antigen (i.e., VEGF, fragment thereof, of a fusion protein containing VEGF). Then, a library is constructed by fusing DNA fragments from the variable regions from an immunized mouse (by RT-PCR and PCR) with a coat protein of the phage. The phages having the desired CDR sequences will bind to the target antigens and can be enriched by bio-panning or ELISA or Dynabeads®, in which the target antigen (e.g., VEGF) is coated on a plate or beads, and the phages are allowed to bind to the antigen. Then, the non-binders are washed away. The bound positive clones are collected and expanded. The panning/enrichment process may be repeated several times to purify the positive clones. The sequences from these positive clones (i.e., the variable sequences) can then be constructed into an antibody framework to produce a full-length construct. The antibodies may be produced from these full-length constructs and purified for assays. The assays may involve in vitro and/or in vivo assays.

In accordance with embodiments of the invention, the phage display approach will be described in details using the following working examples.

### Example 1

### Immunization Procedure

Recombinant human VEGF (from R&D Systems, Inc., Cat. No. 293-VE/CF) was used as an antigen. This antigen was used with Freund's complete adjuvant (FCA) for the initial immunization and Freund's incomplete adjuvant (FIA) or TiterMax for booster injections to immunize mice according a suitable schedule. For example, Table 1 illustrates one exemplary immunization schedule:

TABLE 1
Immunization Scheme

| Schedule | Date | Dose | Adjuvant | Administration |
|---|---|---|---|---|
| Immunized | Week 0 | 15 μg | FCA | s.c. |
| Boost 1st | Week 2 | 10 μg | FIA | s.c. |
| Boost 2nd | Week 4 | 12.5 μg | TiterMax | s.c. |
| Boost 3rd | Week 6 | 10 μg | FIA | s.c. |
| Bleed | Week 7 | | | Test serum titer |
| Boost 4th | Week 8 | 10 μg | FIA | s.c. |

### Example 2

### Testing Serum Titer by ELISA

ELISA plates (e.g., 96-well plates) were coated with a recombinant human VEGF (from R&D Systems, Inc.). Test samples were added to the coated plates and allowed to bind with the coated proteins. After washing to remove the unbound antibodies, the bound antibodies were assessed with a second antibody (e.g., goat anti-mouse IgG coupled with horseradish peroxidase (HRP)). The amounts of bound secondary antibodies can be estimated using a proper substrate for HRP. For example, 3,3′,5,5′-Tetramethylbenzidine (TMB), 3,3′-Diaminobenzidine (DAB), or 2,2′-azino-bis(3-ethylbenzothiazoline-6-sulphonic acid) (ABTS) may be used as a colorimetric substrate of HRP. Table 2 shows results of one example.

TABLE 2
Serum Titers by ELISA (HRP reaction, OD readings)
Phage Display Approach

| Dilution | No. 1 | No. 2 | Normal Sera |
|---|---|---|---|
| 10³x | 2.115 | 2.180 | 0.055 |
| 10⁴x | 1.840 | 2.024 | 0.044 |
| 10⁵x | 0.385 | 0.664 | 0.052 |
| 10⁶x | 0.070 | 0.102 | 0.039 |

Blank: 0.046

### Example 3

### Construction of scFv/Fab Antibody Library

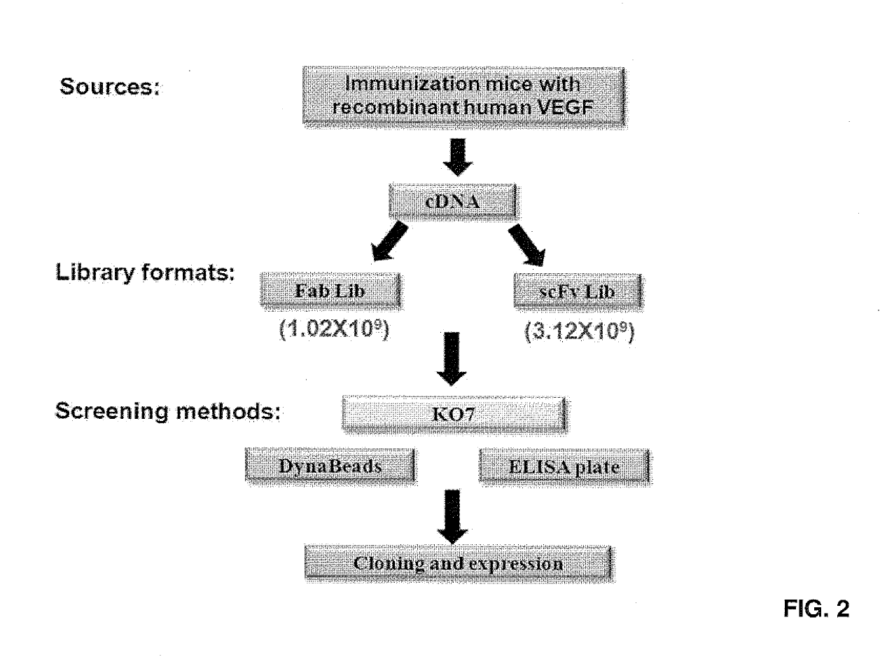

In accordance with embodiments of the invention, antibodies may be generated using phage panning. As shown in FIG. 2, a cDNA library may be constructed from immunized mice. The mice may be immunized, for example, with a recombinant human VEGF (from R&D Systems, Inc.) as described above. The mice were sacrificed and the spleens were removed to extract the total RNA. RT-PCR was then used to obtain antibody fragments (e.g., V_{H}, V_{L}, heavy chain (F_{d}) or light chain) These fragments may be used to construct a Fab library. In addition, these fragments were assembled using PCR to generate antibody cDNA fragments for scFv, which were then used to construct the scFv library. In one example, the Fab library has 1.02×10⁹ diversities and the scFv library has 3.12×10⁹ diversities.

### Example 4

### Preparation of Phages for Screening

The above (scFv or Fab) library stocks each were inoculated into 2×YT medium containing 100 μg/ml ampicillin and 2% glucose (2YTAG) and grown with shaking at 37° C. until the OD (600 nm) reached 0.5. This culture was then infected with M13KO7 or hyper helper phage by adding the helper phage in a ratio of 1:20. The resultant culture was incubated in a 37° C. water bath without shaking for 30 minutes.

Then, the infected cells were collected by spinning at 4,000 rpm for 15 minutes. The cells were resuspended gently in 2×YT containing 100 μg/ml ampicillin and 25 μg/ml kanamycin (2YTAK) and incubated with shaking at 30° C. overnight.

The overnight culture was spun at 10,000 rpm for 20 min to collect the cells. PEG/NaCl (20% PEG 8000, 2.5M NaCl; 1/5 volume) was added to the supernatant. The solution was mixed and left for 1 hour or more at 4° C. It was then spun at 10,000 rpm for 20 min. The supernatant was then aspirated off.

The pellet was resuspended in 40 ml sterile water and the spun at 12,000 rpm for 10 min to remove most of the remaining bacterial debris. A 1/5 volume PEG/NaCl was added to the supernatant again. It was mixed well and left for 1 hr or more at 4° C.

It was again spun at 10,000 rpm for 20 min and the supernatant was aspirated off. The pellet was then resuspended in PBS and spun at 12,000 rpm for 10 min to remove most of the remaining bacterial debris.

The above described is one example for the preparation of phages. This example is for illustration only and not intended to limit the scope of protection. One skilled in the art would appreciate that various modifications and variations are possible. The phages may be screened using ELISA plates or Dynabeads®.

### Example 5

### Selection Using ELISA Plates

ELISA plate (Nunc) was coated with 1 μg/100 μl antigen (e.g., recombinant human VEGF from R&D Systems, Inc.) per well. The antigen coating was performed overnight at 4° C. in PBS, pH 7.4 or 50 mM sodium hydrogen carbonate, pH 9.6. Then, the well were rinsed 3 times with PBS and blocked with 300 μl PBS-5% skim milk (MPBS) per well for 1.5 hours at 37° C. This was followed by rinsing with PBS 3 times.

Then, 100 μl of 10¹¹ to 10¹² phages in 5% MPBS. The solution was incubated for 90 min at 37° C., and the test solution was discarded and washed 3 times with PBS-0.05% Tween20 (PBST).

To each well was added 100 μl PBS. It was incubated for 60 min at 37° C. and washed 3 times with PBST, 1 time with PBS. The excess PBS was shaken out from the plate, and the phages were eluted by adding 100 μl 100 mM triethylamine (TEA) with rotation continuously at 37° C. for 30 min Tris buffer (50 μl, 1 M, pH 7.4) was added to the eluted 100 μl phage, for quick neutralization.

10 ml of an exponentially growing culture of Escherichia coli TG1 was taken and added to 150 μl of the eluted phage. Also 100 μl of the TG1 culture was added to the immunoplate. Both cultures were incubated for 30 min at 37° C. without shaking to allow for infection. Pool the 10 ml and 100 μl of the infected TG1 bacteria were polled and spun at 4000 rpm for 15 min. The pellet of bacteria was resuspended in 2×TY and plate on a large 2YTAG plate. The bacteria were allowed to grow at 30° C. overnight.

### Example 6

### Selection Using Dynabeads®

Dynabeads® were pre-washed with 1 ml PBS three times and resuspended in 2% MPBS. Phage (0.3 ml) was mixed with 0.5 ml 2% PBSM, and the above washed Dynabeads®. The resultant suspension was pre-incubated on a rotator for 30 min.

The Dynabeads® were removed and VEGF (biotin-labeled) was added. The resultant mixture was mixed on a rotator for 90 min. Dynabeads® were pre-washed with 1 ml PBS three times and resuspended in 2% PBSM. This was then incubated on a rotator for 90 min.

The phage-VEGF mix was added to the equilibrated Dynabeads® on a rotator for another 30 min. The Dynabeads® were then washed with 1 ml 0.05% PBST, 2% PBSM, and PBS. The bound phages were then eluted with 1 ml 100 mM TEA. During the incubation, tubes were prepared with 0.5 ml 1M Tris, pH 7.4 to get ready for the addition of the eluted phages for quick neutralization.

6 ml of an exponentially growing culture of TG1 was taken and the TEA eluted phage was added. Also, 4 ml of the E. coli TG1 culture was added to the beads. Both cultures for 30 min at 37° C. (water bath) were incubated without shaking.

The infected TG1 bacterial was pooled and spun at 4000 rpm for 15 min. The pelleted bacterial in 1 ml of 2×YT was resuspended and plated on a large 2TYAG plate. The bacteria were grown at 30° C. overnight.

### Example 7

### Preparation of Next Round Phage

5-6 ml of 2×YT containing 15% glycerol was added to the bacterial plate that had been grown overnight as described above and the colonies were loosen with a glass spreader. 50-100 μl of the scraped bacteria was added to 100 ml of 2×YTAG. The bacteria grew with shaking at 37° C. until the OD at 600 nm is 0.5. 10 ml of this culture with M13KO7 helper phage was infected by adding helper phage in the ratio of 1:20. The infected culture was incubated without shaking in a 37° C.

The infected cells at 4000 rpm for 15 min were spun to collect the bacteria. The pellet was resuspended gently in 50 ml of 2×YTAK and the culture was incubated with shaking at 30° C. overnight.

40 ml of the overnight culture was taken and spun at 10,000 rpm for 20 min to collect the supernatant. 1/5 volume (8 ml) PEG/NaCl was added to the supernatant, mixed well and left it for 1 hr or more at 4° C. The supernatant was spun at 10,000 rpm for 20 min and then aspirated off. The pellet was resuspended in 2 ml PBS and spun at 12000 rpm for 10 min to remove most of the remaining bacterial debris.

### Example 8

### Screening of VEGF-Positive Phage by ELISA

Individual colonies was incubated from the plate into 200 μl 2×YTAG 96-well plates and grew with shaking overnight at 37° C. A 96-well was used as a transfer device to transfer 50 μl inoculum from the plate to a second 96-well plate containing 200 μl of 2×YTAG per well and grew with shaking at 37° C. for 2 hr. 50 μl 2×YTAG with 10⁹ pfu M13KO7 helper phage was added to each well of the second plate, stand for 30 min at 37° C., and then shaken for 1 hr at 37° C.

The plate was spun at 4000 rpm for 30 min, and the supernatant was then aspirated off. The pellet was resuspended in 300 μl 2×YTAK and grew with shaking overnight at 30° C. The mixture was spun at 4000 rpm for 30 min and 100 μl of the culture supernatant was used in phage ELISA.

ELISA plates were coated with 1 μg/100 μl per well of protein antigen. Wells were rinsed for 3 times with PBS and blocked with 300 μl 2% MPBS per well for 2 hr at 37° C. Wells were rinsed for 3 times with PBS. 100 μl phage culture supernatant was added as detailed above and incubated for 90 min at 37° C. The test solution was discarded and washed three times with PBS. An appropriate dilution of HRP-anti-M13 antibody in 2% MPBS was added, incubated for 90 min at 37° C., and washed three times with PBST.

The reaction mixture was developed with substrate solution (TMB). The reaction was stopped by adding 50 μl 1 M sulfuric acid. The color should turn yellow. The OD at 650 nm and at 450 nm was read. Readings were obtained by subtracting OD 650 from OD 450.

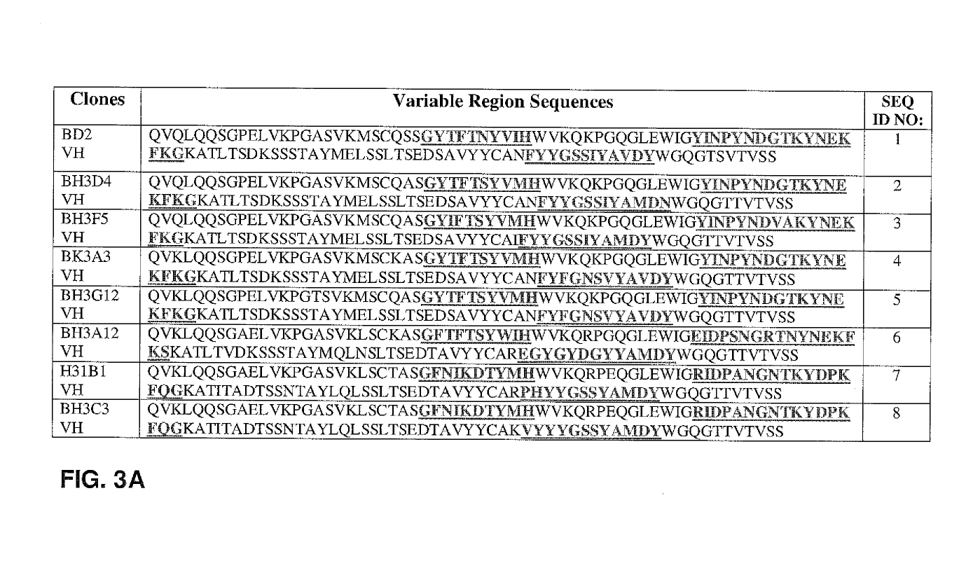

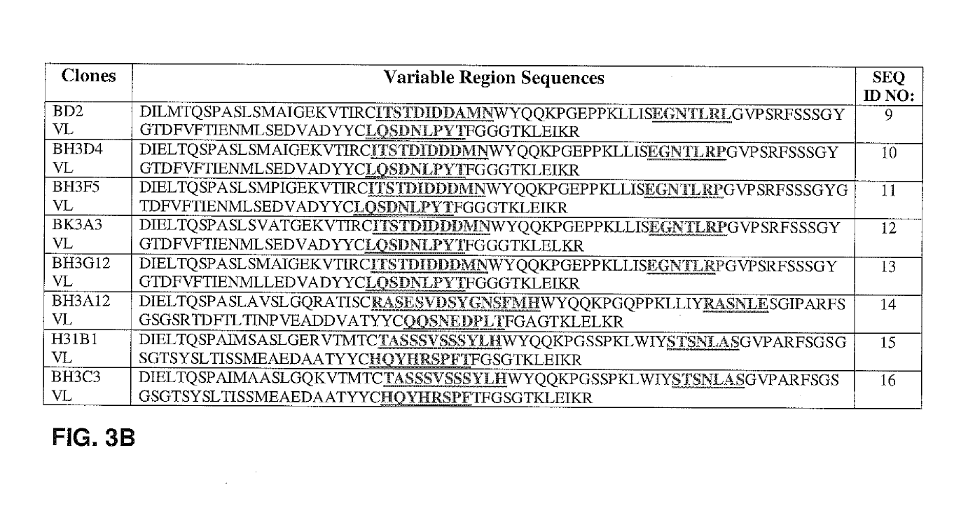

Exemplary results of the bio-panning from both the Dynabeads® and ELISA plate methods are shown in FIGS. 3A and 3B. FIG. 3A shows the sequences for the heavy chain variable regions of these clones (SEQ ID NO: 1, SEQ ID NO: 2, SEQ ID NO: 3, SEQ ID NO: 4, SEQ ID NO: 5, SEQ ID NO: 6, SEQ ID NO: 7, and SEQ ID NO: 8) and FIG. 3B shows the sequences for light chain variable regions of these clones (SEQ ID NO: 9, SEQ ID NO: 10, SEQ ID NO: 11, SEQ ID NO: 12, SEQ ID NO: 13, SEQ ID NO: 14, SEQ ID NO: 15, and SEQ ID NO: 16).

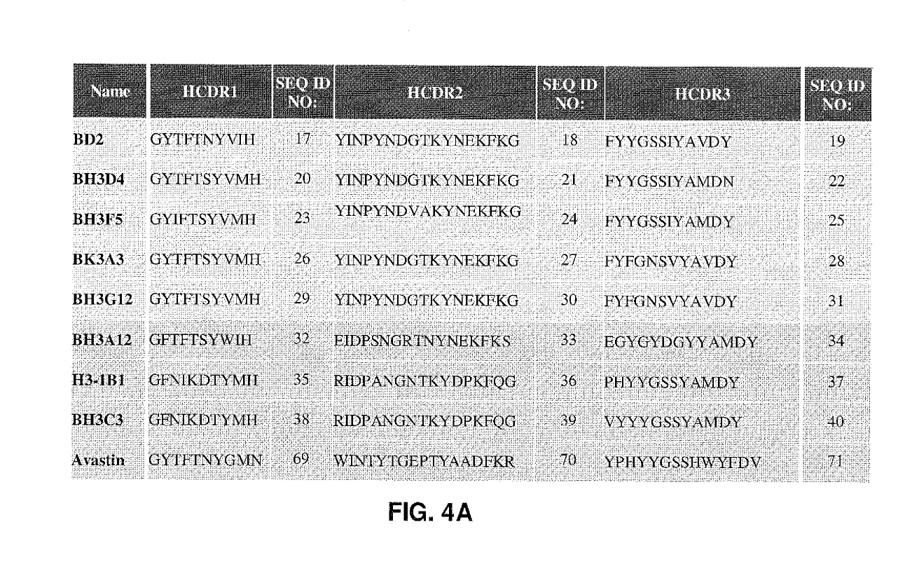

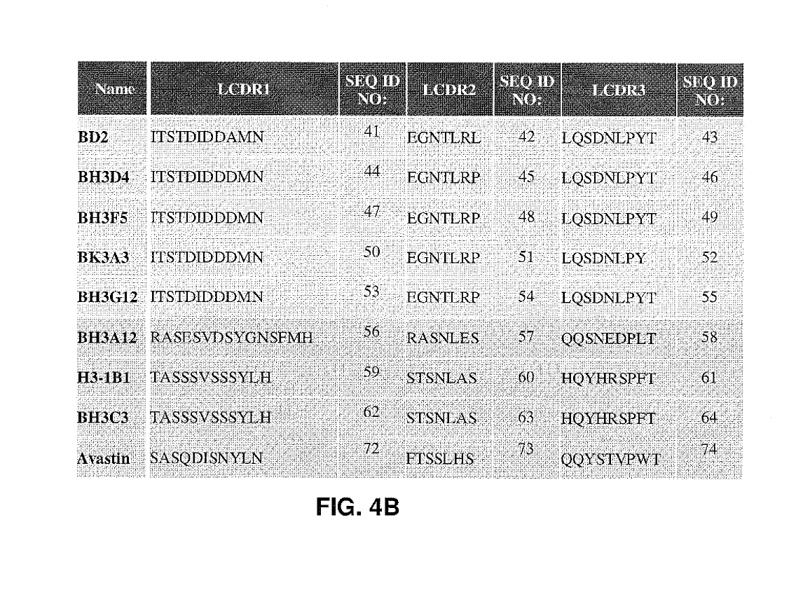

The sequences for the heavy chain and light chain variable regions for these clones have been elucidated and are shown in FIGS. 3A and 3B, respectively. As shown in FIGS. 4 and 4B B, the sequences for the heavy chain CDR1 (i.e., CDRH1) are SEQ ID NO:17, 20, 23, 26, 29, 32, 35, and 38, the sequences for CDRH2 are SEQ ID NO:18, 21, 24, 27, 30, 33, 36, and 39, the sequences for CDRH3 are SEQ ID NO:19, 22, 25, 28, 31, 34, 37, and 40, the sequences for the light chain CDR1 (i.e., CDRL1) are SEQ ID NO: 41, 44, 47, 50, 53, 56, 59, and 62, the sequences for CDRL2 are SEQ ID NO: 42, 45, 48, 51, 54, 57, 60, or 63, and the consensus sequences for CDRL3 are SEQ ID NO: 43, 46, 49, 52, 55, 58, 61, and 64.

One skilled in the art would appreciate that an antibody (or a fragment thereof) containing one or more of the CDRH and/or CDRL sequences (or homologous sequences) can be expected to bind VEGF and may have therapeutic uses. For example, some antibodies or a fragment thereof (e.g., Fab, scFv, F(ab′)2, etc.) may contain a heavy chain or a light chain that comprises one, two or three CDR sequences (or homologous sequences) shown in these clones. Some antibodies or a fragment thereof may contain both a heavy and a light chain that each comprises one or more of these sequences (or homologous sequences). In accordance with embodiments of the invention, a homologous sequence may comprise 50%, 60%, 70%, 80%, 90% or higher sequence identity, as compared to the target sequence. In accordance with embodiments of the invention, a homologous sequence preferably as a sequence identify of 80% or higher, more preferably 90% or higher, and most preferably 95% or higher.

### Example 9

### Expression of Full Length Antibodies

The variable region sequences obtained from phage panning may be inserted into constant regions of an antibody frame work to produce a full-length antibody. For example, the genes encoding the VH and VL chains of anti-VEGF antibodies may be inserted into expression vectors that contain the constant regions of an antibody. FreeStyle™ 293 cells were transfected with the vector thus constructed. The following procedures are used to transfect the vector thus constructed into suspensions of FreeStyle™ 293 cells in a 30 ml volume. The cells may be kept in FreeStyle™ 293 Expression Medium during transfection.

Approximately 24 hrs before transfection, the FreeStyle™ 293 cells were passed at 2×10⁶ cells/ml for 15 ml. The flask(s) was placed in an incubator at 37° C. containing 8% CO₂. Then, 37.5 μg of plasmid DNA was diluted into 1.5 ml sterile 150 mM NaCl to a total volume of 1.5 ml. In a separate tube, 37.5 μl of PEI (2.0 mg/ml) was diluted in 1.5 ml sterile 150 mM NaCl.

The DNA and PEI solutions were allowed to sit at room temp for 5 minutes. The solutions were mixed gently by inverting the tubes and then allowed the tubes to stand at room temp for around 10-20 minutes. DNA-PEI mixture was added into F293 cells and incubated the transfected cell on an orbital shaker platform rotating at 135-150 rpm at 37° C., 8% CO₂ in an incubator for 4 hours. Then, an equal volume of fresh culture medium was added to a total volume of 30 ml, and the cells were cultured for 5-7 days. Cells are then harvested for protein purification and quantification.

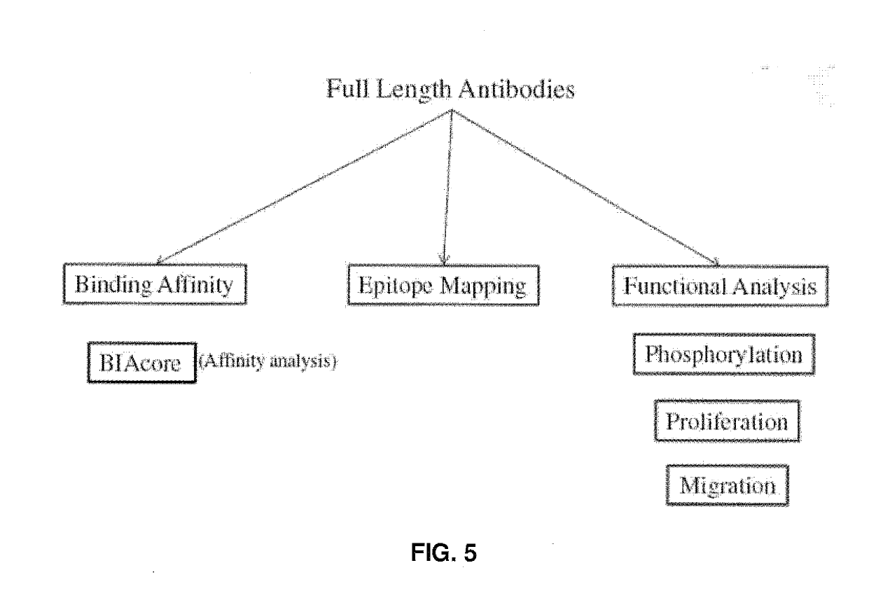

As shown in FIG. 5, the purified full-length antibodies may be analyzed for their binding affinities (e.g., using BIAcore or any other suitable methods). The full-length antibodies, or the fragments thereof, may be used to map the epitopes on VEGF. The full-length antibodies may also be analyzed for their functions, such as their effects on the VEGF-induced receptor phosphorylation or VEGF-induced cell proliferation or migration.

### Example 10

### Affinity Measurements and Kinetic Analysis Using BIAcore

For use as therapeutic agents, antibodies should preferably have good affinities to the target molecule (e.g., VEGF). The affinities and kinetics of various antibodies binding to VEGF may be assessed using any suitable instrument, such as surface plasmon resonance (SPR)-based assay on BIAcore T100. For example, the binding kinetics were measured and analyzed by multi-cycle kinetics (MCK) methods using the associated software.

As an example, human IgG (Fc) antibody was immobilized on CM5 chips at a density that allowed one to achieve Rₘₐₓ in the range of 50-150 Response Units (RU).

In this example, the kinetic assay parameters were as follows: data collection rate 1 Hz; dual detection mode; temperature: 25° C.; concentration unit: nM; and buffer A HBS-EP. The measurements were performed with 5 replicates. The various instrument settings are as follows.

Select Capture, flow path 2-1, chip CM5, regeneration 1.

Select the Ligand Capture and set the parameters as followed, Contact time: 12s, Flow rate: 10 μL/min, Stabilization period: 90s. (In the range of 50-150 Response Units (RU)) (Anti-VEGF antibodies of BD2 and mutants as ligands).

Select the Sample and set the parameters as followed, Contact time: 300s, Flow rate: 40 μL/min, Dissociation time: 500s.

Select the Regeneration and set the parameters as followed, Regeneration solution: 25 mM Glycine pH1.5, Contact time: 60s, Flow Rate: 30 μL/min, Stabilization period: 120s.

Serial dilutions of VEGF with the running buffer (HBS-EP+). The series concentrations obtained are 10, 5, 2.5, 1.25, 0.625, 0.3125, 0.15625, 0 and 1.25 nM (repeat). Prepare and position samples according to Rack Positions.

The results were evaluated with the BIAcoreT100 evaluation software. The binding responses were corrected for buffer effects by subtracting responses from a blank flow cell. A 1:1 Langmuir fitting model was used to estimate the kₐ or kₒₙ (association rate or on-rate) and k_{d} or k_{off} (dissociation rate or off-rate). The K_{D} (or K_{d}) values may be determined from the ratios of k_{off} and kₒₙ (i.e., K_{D}=k_{off}/kₒₙ). Alternatively, the dissociation constants (K_{d} values) may be estimated from the steady-state bound form concentration as a function of the antibody concentrations based on an equilibrium kinetics similar to the Michaelis-Menton equation, and the on rate (kₒₙ) can be estimated from the curved portions from the binding pregress by fitting a first-order reaction kinetics. (The reaction is first order because one of the reagents is held at a constant concentration.) Then, the k_{off} rates may be derived from K_{D} and kₒₙ. Alternatively, k_{off} may be obtained from the exponential decay of the complex as the complex immobilized on the probe is being washed.

### Example 11

### Epitope Mapping

In order to elucidate the residues involved in the biding of the antibodies to VEGF, epitope mapping experiments were performed. Specifically, alanine-scanning method was used to identify residues on VEGF that are critical for antibody binding. The kinetics and affinities of these VEGF mutants were assessed in the same manners described above. The assays can be performed with various mutants in combination with various antibodies. Results from these binding studies would allow one to identify not only critical residues on VEGF, but also important residues in the CDR's.

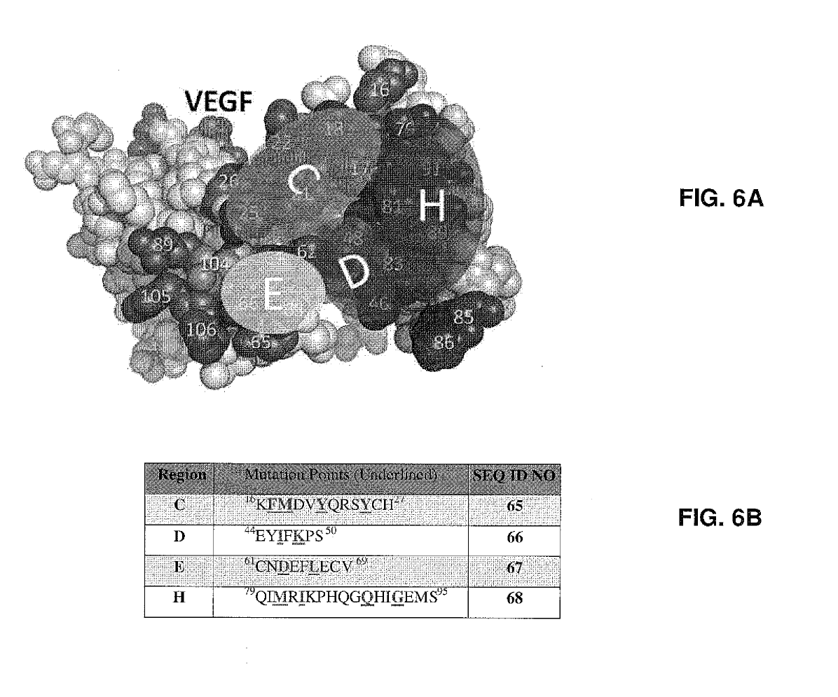

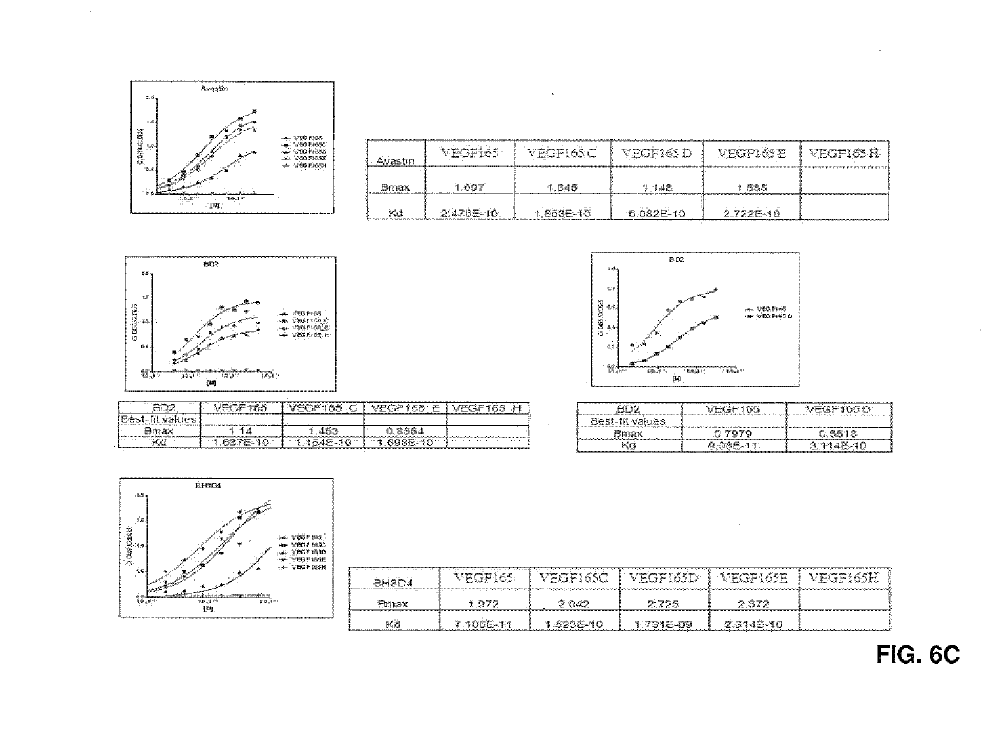

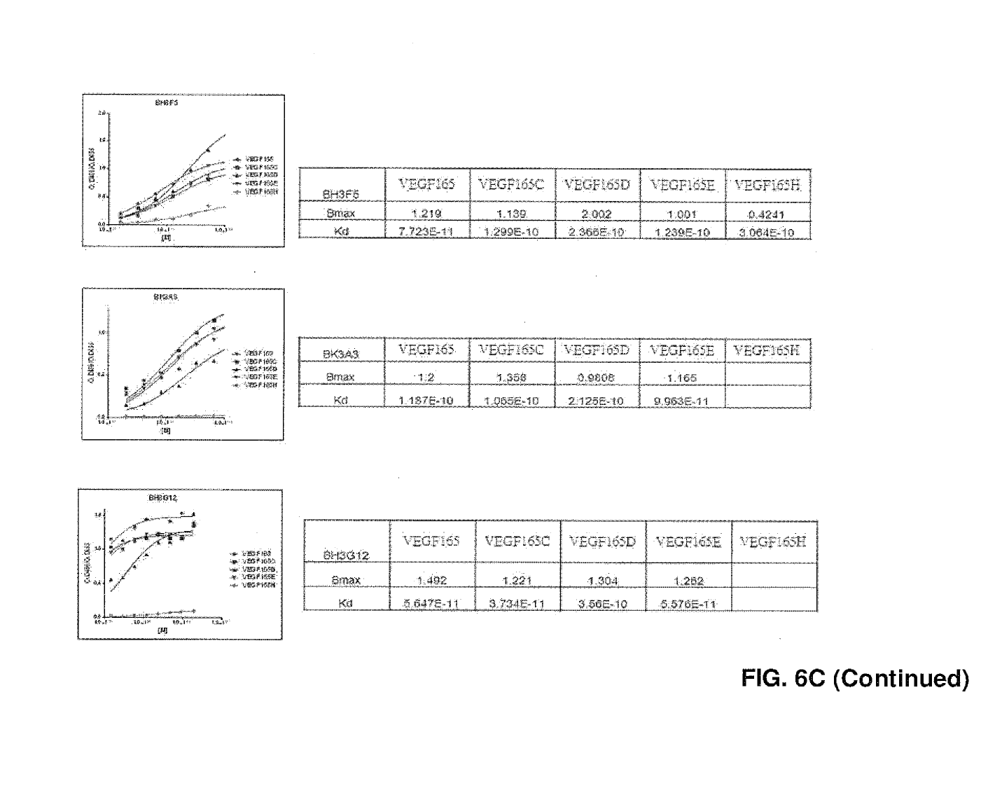

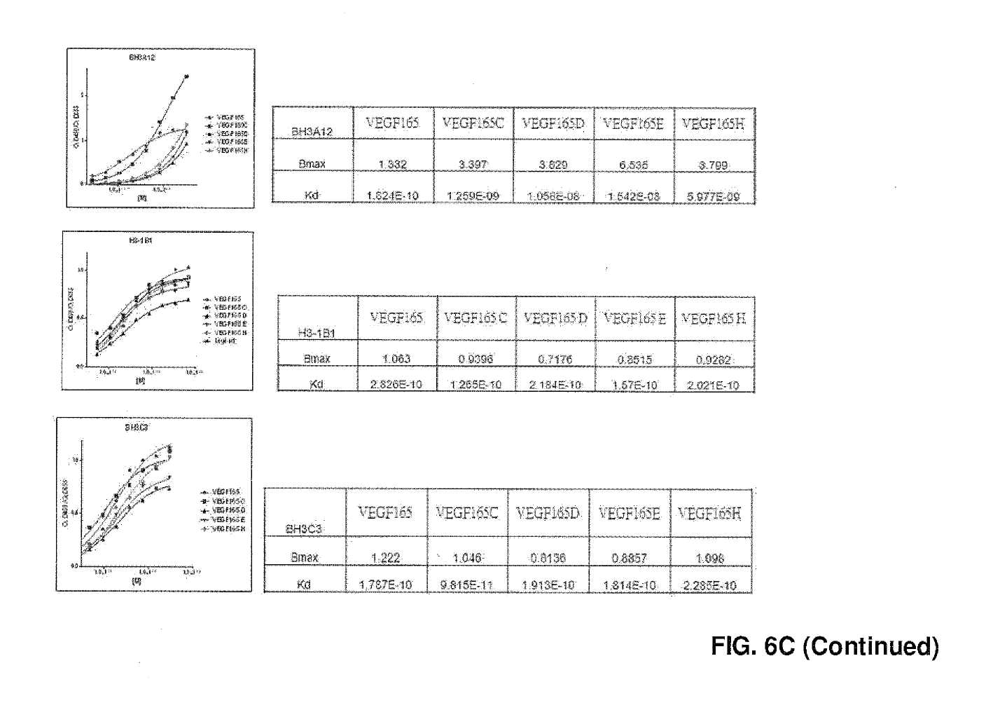

VEGF binds to its receptors to exert its biological functions. The binding face on VEGF has been elucidates. FIG. 6A shows a three-dimensional structure of VEGF₁₆₅ with it receptor (VEGF receptor II) binding face shown. For an anti-VEGF antibody to be useful in clinics, the antibody likely would bind to the same face or nearby such that it would compete or interfere with VEGF binding to its receptors.

FIG. 6A shows four regions (C, D, E, and H) chosen for preparing mutants to map the epitopes on VEGF₁₆₅. The sequences in these four regions (SEQ ID NO: 65-68) are shown in FIG. 6B. The approach is to use alanine substitutions to see which resides in VEGF₁₆₅ are important for binding with the antibodies. The various alanine substitution mutants may be produced with side-directed mutagenesis techniques commonly known in the art. The mutants can be incorporated into expression vectors and transfected into suitable bacterial, yeast, or mammalian cells to produce the mutant VEGF. These protein expression techniques are well-known in the art. Based on these studies, the various residues that are important for antibody bindings, as revealed by alanine replacements, in the four regions are underlined in the sequences shown in FIG. 6B.

The four variants of VEGF₁₆₅ (i.e., with alanine replacements of the underlined residues in FIG. 6B) are referred to as VEGF165C (F17A, M18A, Y21A, and Y25A), VEGF165D (I46A, K48A), VEGF165E (D63A, L66A), and VEGF165H (M81A, I83A, Q89A, G92A).

These variants of VEGF₁₆₅, together with the native VEGF₁₆₅, were used to probe the bindings of various antibodies to the epitopes on VEGF₁₆₅, as described below.

### Example 12

### Epitope Mapping Analysis

The binding assays can be performed in a manner similar to those described above using Dynabeads® or ELISA plates.

Briefly, ELISA plates were coated with 100 ul per well of the natural VEGF₁₆₅ or the above-mentioned variants of VEGF₁₆₅ overnight at 4° C. The wells were rinsed 3 times with PBS, by flipping over the ELISA plates to discard excess liquid, and blocked with 300 ul per well of 5% MPBS for 2 hr at 37° C. The wells were then rinsed again 3 times with PBS.

Add 100 ul each of test anti-VEGF antibodies, in 2-fold serial dilutions. Avastin® was used as a positive control. Then, incubate the plates for 90 min at 37° C. Discard the test solution and wash the wells 3 times with PBS.

Add appropriate dilution of HRP-anti-Human IgG antibody in 5% MPBS (1:10000) to each well. Incubate for 60 min at 37° C., and then wash the wells 3 times with PBS.

Develop with substrate solution TMB 100 ul. Stop the reaction by adding 50 ul of 1 M sulfuric acid. The color should turn yellow. Read the OD at 650 nm and at 450 nm. Subtract OD₆₅₀ readings from OD₄₅₀ readings to obtain values that indicate the extents of antibody bindings.

FIG. 6C shows results of the binding assays for the various antibodies with the native VEGF₁₆₅ and the four VEGF₁₆₅ variants, i.e., VEGF165C, VEGF165D, VEGF165E, and VEGF165H.

With Avastin® (the positive control), the binding to VEGF165C, VEGF165D, and VEGF165E variants remain substantially unchanged, suggesting that the binding site for Avastin® is not located in these regions. However, the binding between Avastin® and VEGF165H was completely abolished, indicating that Avastin® binding site is located in this region and alanine substitutions for the residues shown in SEQ ID NO:68 compromised the binding. These results indicate that the binding site (epitope) for Avastin® on VEGF₁₆₅ is located in the H region, i.e., SEQ ID NO:68.

Antibodies BD2, BK3A3, and BH3G12 behaved in a manner similar to Avastin®—i.e., no significant loss of bindings to VEGF165C, VEGF165D, and VEGF165E, but their bindings with VEGF165H were substantially lost. These results indicate that the binding sites (epitope) for antibodies BD2, BK3A3, and BH3G12 are also located in region H, i.e., SEQ ID NO: 68.

On the other hand, the epitopes for antibodies BH3D4, BH3A12, BH3F5, H3-1B1 and BH3C3 are distinct from Avastin®. The epitope for antibody BH3D4 may involve two regions (D and H), while the epitope for antibody BH3A12 may involve antibodies are not located in these four regions (C, D, E, and H).

Based on these results, one can conclude that the above antibodies bind to epitopes in regions D, E, and/or H on VEGF, or they bind to epitopes outside of the C, D, E, and H regions. However, none of the antibodies bind to region C.

### Example 13

### VEGF-Induced HUVEC Proliferation Assay

VEGF is known for its angiogenic property. It can bind to VEGF receptors to cause receptor phosphorylation, leading to signal transduction that may result in new blood vessel formation. New blood vessel formation (angiogenesis) involves proliferation of vascular endothelial cells, such as human vescular endothelial cells (HUVEC). Thus, to test whether the antibodies have the abilities to block the VEGF function, one may test whether the antibodies can prevent VEGF-induced HUVEC proliferation. This assay may be performed as follows:

Avastin (as a positive control) or the antibody test samples are diluted with 10% FBS-DMEM to generate a series of different concentrations, e.g., from 800 ng/ml, 2 fold serial dilutions. One hundred (100) μl of antibody solution is added to each well of a 96-well plate. The test may be run in duplicate or triplicate.

CHO hVEGF is diluted with Medium 200 (which is a sterile, liquid medium for the culture of human large vessel endothelial cells available from Life Technologies, Grand Island, N.Y.) to 50 ng/ml. Add 50 μl CHO hVEGF to each well on the plate. The plate is then incubated at room temperature for 30 mins.

After the incubation, the cells are harvested and then resuspended in Medium 200 to 8×10⁴ cells/ml. An aliquot of 50 μl of the cell culture is added to each well on the plate. The plate is incubated for 96 hours at 37° C. in a 5% CO₂ incubator.

After the incubation, 20 μl WST-1 cell proliferation assay reagent (available from Cell Biolabs, Inc., San Diego, Calif.) is added to each well. The plate is incubated for 4 hours at 37° C. in a 5% CO₂ incubator. Then, the absorbance at 450 nm and 655 nm are measured to derive OD difference (A₄₅₀ₙₘ-A₆₅₅ₙₘ using an ELISA reader.

### Example 14

### VEGF-Induced VEGFR2 Phosphorylation Assay

As noted above, binding of VEGF to its receptor cause receptor phosphorylation. Therefore, another way of assaying the activities of the antibodies is to assay their abilities to inhibit such receptor phosphorylation. One method for this assay is as follows:

HUVEC cells are grown to 90% confluence with growth factor starvation (2% FBS) at 37° C. for 16 hours in a 6-well dish. The mixtures including VEGF (10 ng/ml) and various amounts of antibody (50, 10, 2, 0.4, 0.08 nM) are prepared and incubated at 37° C. for 30 mins

After 16 hours starvation, remove the medium and incubate the HUVEC cells with the prepared mixtures of various amounts of antibody solution at RT for 10 mins.

The cells are washed with 2 ml PBS (with 1× phosphatase inhibitor) once. Then, 150 ul 1× sampling buffer (with 1× Phosphatase inhibitor) is added to lysis the cells.

The cells are boiled at 100° C. for 10 mins Aliquots (25 ul) of cell lysate are run on 6% SDS-PAGE. Protein bands are transferred to a PVDF membrane, which had been immersed in fresh methanol prior to use, at 100 V, 4° C. for 70 mins.

The membrane was blocked with 5% milk in TBST buffer or 1^{st} antibody at 4° C. overnight. The membrane is then blotted (Western blot) with anti-VEGFR2 (1:1000 dilution) and anti-phospho-VEGFR2 (Tyr1175)(1:1000 dilution).

The membrane is washed with TBST (0.05% tween 20) at RT 10 mins three times, and then the second anti-rabbit IgG-HRP (1:5000 dilution) is added at RT for an hour.

The membrane is again washed with TBST (0.05% tween 20) at RT 10 mins three times. Then, it is stained with femto ECL (enhanced chemiluminescence).

### Example 15

### VEGF-Induced HUVEC Migration Assay

As noted above, binding of VEGF to its receptor cause receptor phosphorylation, leading to signal transduction and HUVEC proliferation. To form new blood vessels, the HUVEC cells migrate to form new tubular structures. Therefore, another way of assaying the activities of the antibodies is to assay their abilities to inhibit such HUVEC migration. One method for this assay is as follows:

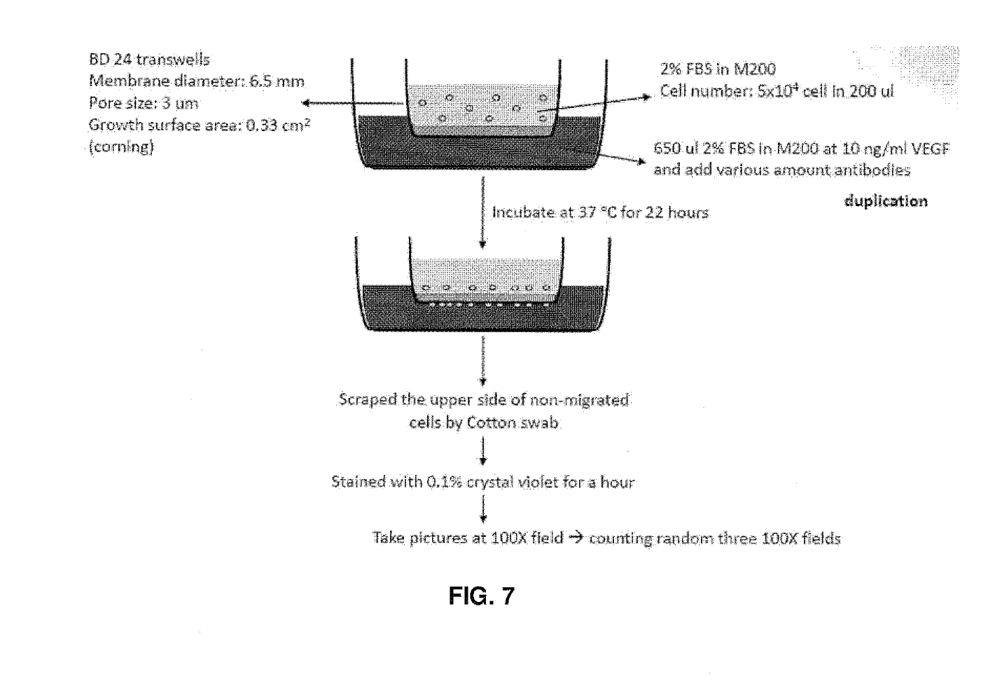

To investigate the effects of anti-VEGF antibodies on HUVEC migration, the migration activity of HUVEC cells was assessed using a Transwell assay system, which has two chambers separated by a membrane having micropores (e.g., 3 μm pores; Becton Dickinson, Franklin Lakes, N.J.). As illustrated in FIG. 7, HUVEC cells are grown in the upper chamber in Medium 200 containing 2% FBS. The cell number in this example is 5×10⁴ cells in 200 μl. In the lower chamber is placed 650 μl Medium 200 with 2% FBS, 10 ng/ml VEGF, and various concentrations of the test antibodies.

The cells are incubated at 37° C. for 22 hours. After the incubation period, the upper side of the membrane is scraped to remove the non-migrated cells using cotton swab. Then, the cells on the lower side of the membrane are counted under microscope at 100× magnification. The cell counts are obtained from three random fields. Data are presented as mean±SD. T-test is used to compare activity between each group. The P values<0.05 are considered statistically significant.

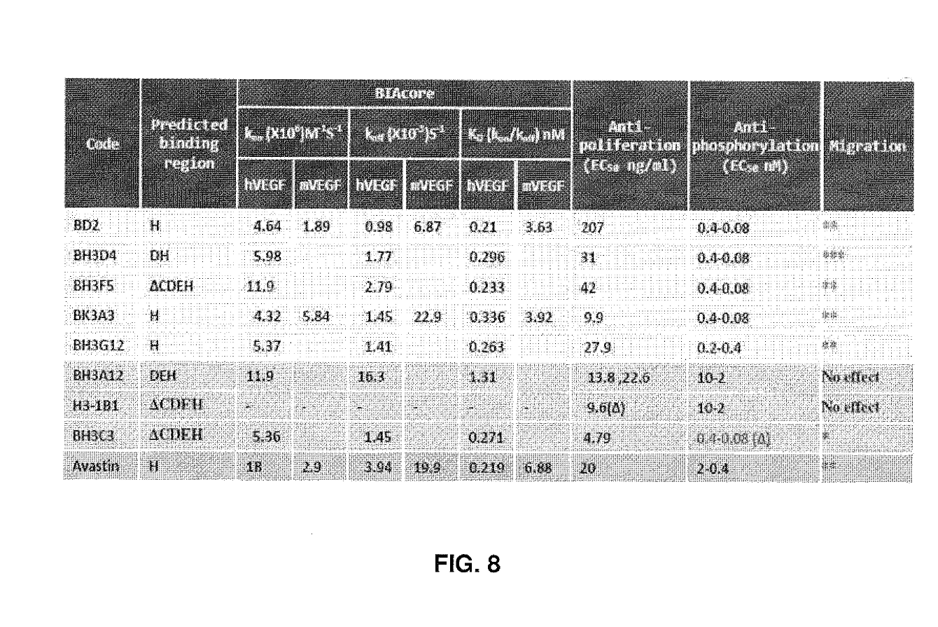

FIG. 8 summaries the results from the BIAcore binding assays and three different VEGF inhibition assays (described below). Results from Avastin® are also shown for comparison. As can be seen from the comparison, several of the clones exhibit activities similar to those of Avastin®. For example, human VEGF binding affinities for BH3C3 (0.271 nM), BH3G12 (0.263 nM), BK3A3 (0.336 nM), BH3F5 (0.233 nM), BH3D4 (0.296 nM), and BD2 (0.21 nM) are similar to that of Avastin® (0.219 nM).

### Example 16

### In Vivo Tumor Growth Inhibition Assay in Xenograft Model

As noted above, VEGF is essential for tumor growth and metastasis. The above in vitro assays prove that the antibodies of the present invention are effective in inhibiting various biological function of VEGF. Therefore, one would expect that these antibodies should also be effective in inhibiting tumor growth and metastasis in vivo. To test these effects in vivo, a tumor xenograft model in mice was used.

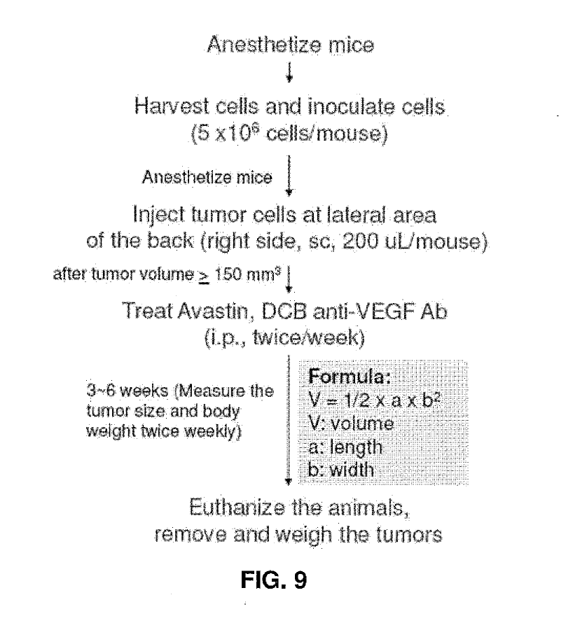

A method for this in vivo assay is illustrated in FIG. 9. As shown, mice were anesthetized, and then tumor cells were injected at lateral area of the back of the anesthetized mice (right side, sc, 200 μl/mouse).

After the tumors have grown to a volume ≧150 mm³, the mice were treated with Avastin® (as a positive control) and the test anti-VEGF antibodies at 0.1 mg/kg, as shown in FIG. 9. The antibodies were injected twice per week.

For the following 3-6 weeks, the tumor sizes and body weights of the mice were measured twice per week. At the end of the test, the mice were sacrificed and the tumors are removed and weighed. The tumor volume was calculated as: V=1/2×a×b², wherein V is the volume, a and b are length and width, respectively.

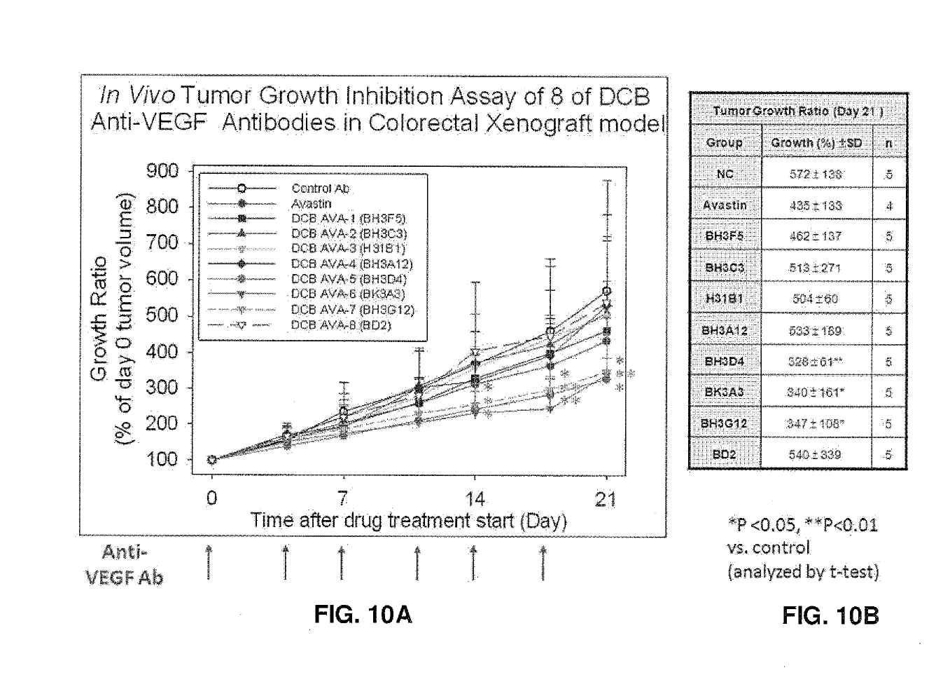

FIG. 10A shows results of tumor growth over the test period (21 days) for various treatment groups (one blank control group, Avastin® group, and eight different anti-VEGF groups, BD2, BH3G12, BK3A3, BH3D4, BH3A12, H31B1, BH3C3, and BH3F5). The results are summarized in the Table shown in FIG. 10B, in which growth ratios (% of day 0 tumor volume) for NC (control), Avastin®, and the 8 antibodies are shown. As seen from the data in the Table of FIG. 10B, all eight anti-VEGF antibodies are effective in reducing tumor growth in the in vivo xenograft model. Compared with Avastin®, BH3D4, BK3A3, and BH3G12 are more effective in inhibiting tumor growths.

### Treatment Methods

As noted above, antibodies of the invention are effective in inhibiting VEGF functions. Therefore, these antibodies may be used as therapeutic agents for the treatment of VEGF-related disorders, such as diabetic retinopathy and cancers. In accordance with embodiments of the invention, the antibodies may be monoclonal antibodies. Such antibodies may be humanized antibodies or human antibodies. In accordance with embodiments of the invention, a subject in need of such treatment or prevention will be given an effective amount of the antibody.

“Treating” or “treatment” refers to administration of an antibody or composition thereof to a subject, who has one or more of the above-descried disorders, with the purpose to cure, alleviate, relieve, remedy, prevent, or ameliorate the disorder, the symptom of the disorder, the disease state secondary to the disorder, or the predisposition toward the disorder. “Preventing” or “prevention” refers to eliminating or reducing the occurrence of the above described disorders. As understood in the art, “prevent” or “prevention” does not require complete (100%) avoidance of the occurrence of such disorders. Instead, reduction in the probability or extents of the disorders would be considered successful prevention.

An “effective amount” refers to an amount that is capable of producing a medially desirable result in a treated subject. The treatment method can be performed alone or in conjunction with other drugs or therapy. For treatment of a skin disorder, such as SJS and TEN, the therapeutic agent may be delivered topically or internally (e.g., by injection).

The dosage required depends on the choice of the route of administration; the nature of the formulation; the nature of the patient's illness; the subject's size, weight, surface area, age, and sex; other drugs being administered; and the judgment of the attending physician. Suitable dosages are in the range of 0.01-100 mg/kg. Variations in these dosage levels can be adjusted using standard empirical routines for optimization as is well understood in the art. Encapsulation of the therapeutic agent in a suitable delivery vehicle (e.g., polymeric microparticles or implantable devices) may increase the efficiency of delivery.

The above examples demonstrate various aspect and utility of embodiments of the invention. While the invention has been described with respect to a limited number of embodiments, those skilled in the art, having benefit of this disclosure, will appreciate that other embodiments can be devised which do not depart from the scope of the invention as disclosed herein. Accordingly, the scope of the invention should be limited only by the attached claims.

1. An anti-VEGF antibody, or a binding fragment thereof, comprising:
a heavy-chain variable region that comprises: (1) a CDRH1 sequence selected from SEQ ID NO:17, 20, 23, 26, 29, 32, 35, or 38), (2) a CDRH2 sequence selected from SEQ ID NO:18, 21, 24, 27, 30, 33, 36, or 39, and (3) a CDRH3 sequence selected from SEQ ID NO:19, 22, 25, 28, 31, 34, 37, or 40, wherein the antibody has the ability to neutralize an activity of VEGF.

2. An anti-VEGF antibody, or a binding fragment thereof, comprising:
a light-chain variable region that comprises: (1) a CDRL1 sequence selected from SEQ ID NO: 41, 44, 47, 50, 53, 56, 59, or 62, (2) a CDRL2 sequence selected from SEQ ID NO: 42, 45, 48, 51, 54, 57, 60, or 63, and (3) a CDRL3 sequence selected from SEQ ID NO: 43, 46, 49, 52, 55, 58, 61, or 64, wherein the antibody has the ability to neutralize an activity of VEGF.

3. The anti-VEGF antibody, or the binding fragment thereof, according to claim 2, further comprising:
a heavy-chain variable region that comprises: (1) a CDRH1 sequence selected from SEQ ID NO:17, 20, 23, 26, 29, 32, 35, or 38), (2) a CDRH2 sequence selected from SEQ ID NO:18, 21, 24, 27, 30, 33, 36, or 39, and (3) a CDRH3 sequence selected from SEQ ID NO:19, 22, 25, 28, 31, 34, 37, or 40.

4. The anti-VEGF antibody, or the binding fragment thereof, according to claim 1, wherein the heavy-chain variable region and the light-chain variable region of the antibody, respectively, comprise the sequences of:
SEQ ID NO: 1 and SEQ ID NO: 9, or
SEQ ID NO: 2 and SEQ ID NO: 10, or
SEQ ID NO: 3 and SEQ ID NO: 11, or
SEQ ID NO: 4 and SEQ ID NO: 12, or
SEQ ID NO: 5 and SEQ ID NO: 13, or
SEQ ID NO: 6 and SEQ ID NO: 14, or
SEQ ID NO: 7 and SEQ ID NO: 15, or
SEQ ID NO: 8 and SEQ ID NO: 16.

5. The anti-VEGF antibody, or the binding fragment thereof, according to claim 1, wherein the antibody binds to one or more of the sequences of SEQ ID NO:66, SEQ ID NO:67, and SEQ ID NO:68.

6. The anti-VEGF antibody, or the binding fragment thereof, according to claim 1, wherein the antibody is a monoclonal antibody.

7. The anti-VEGF antibody, or the binding fragment thereof, according to claim 1, wherein the antibody is a humanized antibody or a human antibody.

8. A method for treating or preventing VEGF-related disorder using the anti-VEGF antibody, or the binding fragment thereof, according to claim 1, the method comprising: administering to a subject in need thereof an effective amount of the antibody or the binding fragment.

9. The method of claim 8, wherein the VEGF-related disorder is abnormal angiogenesis or tumor growth.

10. The method of claim 8, wherein the VEGF-related disorder is diabetic retinopathy or age-related macular degeneration.

11. The anti-VEGF antibody, or the binding fragment thereof, according to claim 2, wherein the heavy-chain variable region and the light-chain variable region of the antibody, respectively, comprise the sequences of:
SEQ ID NO: 1 and SEQ ID NO: 9, or
SEQ ID NO: 2 and SEQ ID NO: 10, or
SEQ ID NO: 3 and SEQ ID NO: 11, or
SEQ ID NO: 4 and SEQ ID NO: 12, or
SEQ ID NO: 5 and SEQ ID NO: 13, or
SEQ ID NO: 6 and SEQ ID NO: 14, or
SEQ ID NO: 7 and SEQ ID NO: 15, or
SEQ ID NO: 8 and SEQ ID NO: 16.

12. The anti-VEGF antibody, or the binding fragment thereof, according to claim 3, wherein the heavy-chain variable region and the light-chain variable region of the antibody, respectively, comprise the sequences of:
SEQ ID NO: 1 and SEQ ID NO: 9, or
SEQ ID NO: 2 and SEQ ID NO: 10, or
SEQ ID NO: 3 and SEQ ID NO: 11, or
SEQ ID NO: 4 and SEQ ID NO: 12, or
SEQ ID NO: 5 and SEQ ID NO: 13, or
SEQ ID NO: 6 and SEQ ID NO: 14, or
SEQ ID NO: 7 and SEQ ID NO: 15, or
SEQ ID NO: 8 and SEQ ID NO: 16.

13. The anti-VEGF antibody, or the binding fragment thereof, according to claim 2, wherein the antibody binds to one or more of the sequences of SEQ ID NO:66, SEQ ID NO:67, and SEQ ID NO:68.

14. The anti-VEGF antibody, or the binding fragment thereof, according to claim 3, wherein the antibody binds to one or more of the sequences of SEQ ID NO:66, SEQ ID NO:67, and SEQ ID NO:68.

15. The anti-VEGF antibody, or the binding fragment thereof, according to claim 4, wherein the antibody binds to one or more of the sequences of SEQ ID NO:66, SEQ ID NO:67, and SEQ ID NO:68.

16. The anti-VEGF antibody, or the binding fragment thereof, according to claim 11, wherein the antibody binds to one or more of the sequences of SEQ ID NO:66, SEQ ID NO:67, and SEQ ID NO:68.

17. The anti-VEGF antibody, or the binding fragment thereof, according to claim 12, wherein the antibody binds to one or more of the sequences of SEQ ID NO:66, SEQ ID NO:67, and SEQ ID NO:68.

18. The method of claim 8, wherein the anti-VEGF antibody, or the binding fragment thereof, further comprises a light-chain variable region that comprises: (1) a CDRL1 sequence selected from SEQ ID NO: 41, 44, 47, 50, 53, 56, 59, or 62, (2) a CDRL2 sequence selected from SEQ ID NO: 42, 45, 48, 51, 54, 57, 60, or 63, and (3) a CDRL3 sequence selected from SEQ ID NO: 43, 46, 49, 52, 55, 58, 61, or 64, wherein the antibody has the ability to neutralize an activity of VEGF.

19. The method of claim 8, wherein the heavy-chain variable region and the light-chain variable region of the antibody, respectively, comprise the sequences of:
SEQ ID NO: 1 and SEQ ID NO: 9, or
SEQ ID NO: 2 and SEQ ID NO: 10, or
SEQ ID NO: 3 and SEQ ID NO: 11, or
SEQ ID NO: 4 and SEQ ID NO: 12, or
SEQ ID NO: 5 and SEQ ID NO: 13, or
SEQ ID NO: 6 and SEQ ID NO: 14, or
SEQ ID NO: 7 and SEQ ID NO: 15, or
SEQ ID NO: 8 and SEQ ID NO: 16.

20. The method of claim 8, wherein the antibody binds to one or more of the sequences of SEQ ID NO:66, SEQ ID NO:67, and SEQ ID NO:68.


SEQUENCE LISTING (74 sequences)

SEQ ID NO: 1 - protein, 121 aa, Mus musculus
QVQLQQSGPELVKPGASVKMSCQSSGYTFTNYVIHWVKQKPGQGLEWIGYINPYNDGTKYNEKFKGKATLTSDKSSSTAYMELSSLTSEDSAVYYCANFYYGSSIYAVDYWGQGTSVTVSS

SEQ ID NO: 2 - protein, 121 aa, Mus musculus
QVQLQQSGPELVKPGASVKMSCQASGYTFTSYVMHWVKQKPGQGLEWIGYINPYNDGTKYNEKFKGKATLTSDKSSSTAYMELSSLTSEDSAVYYCANFYYGSSIYAMDNWGQGTTVTVSS

SEQ ID NO: 3 - protein, 121 aa, Mus musculus
QVQLQQSGPELVKPGASVKMSCQASGYIFTSYVMHWVKQKPGQGLEWIGYINPYNDVAKYNEKFKGKATLTSDKSSSTAYMELSSLTSEDSAVYYCAIFYYGSSIYAMDYWGQGTTVTVSS

SEQ ID NO: 4 - protein, 121 aa, Mus musculus
QVKLQQSGPELVKPGASVKMSCKASGYTFTSYVMHWVKQKPGQGLEWIGYINPYNDGTKYNEKFKGKATLTSDKSSSTAYMELSSLTSEDSAVYYCANFYFGNSVYAVDYWGQGTTVTVSS

SEQ ID NO: 5 - protein, 121 aa, Mus musculus
QVKLQQSGPELVKPGTSVKMSCQASGYTFTSYVMHWVKQKPGQGLEWIGYINPYNDGTKYNEKFKGKATLTSDKSSSTAYMELSSLTSEDSAVYYCANFYFGNSVYAVDYWGQGTTVTVSS

SEQ ID NO: 6 - protein, 122 aa, Mus musculus
QVKLQQSGAELVKPGASVKLSCKASGFTFTSYWIHWVKQRPGQGLEWIGEIDPSNGRTNYNEKFKSKATLTVDKSSSTAYMQLNSLTSEDTAVYYCAREGYGYDGYYAMDYWGQGTTVTVSS

SEQ ID NO: 7 - protein, 121 aa, Mus musculus
QVKLQQSGAELVKPGASVKLSCTASGFNIKDTYMHWVKQRPEQGLEWIGRIDPANGNTKYDPKFQGKATITADTSSNTAYLQLSSLTSEDTAVYYCARPHYYGSSYAMDYWGQGTTVTVSS

SEQ ID NO: 8 - protein, 121 aa, Mus musculus
QVKLQQSGAELVKPGASVKLSCTASGFNIKDTYMHWVKQRPEQGLEWIGRIDPANGNTKYDPKFQGKATITADTSSNTAYLQLSSLTSEDTAVYYCAKVYYYGSSYAMDYWGQGTTVTVSS

SEQ ID NO: 9 - protein, 108 aa, Mus musculus
DILMTQSPASLSMAIGEKVTIRCITSTDIDDAMNWYQQKPGEPPKLLISEGNTLRLGVPSRFSSSGYGTDFVFTIENMLSEDVADYYCLQSDNLPYTFGGGTKLEIKR

SEQ ID NO: 10 - protein, 108 aa, Mus musculus
DIELTQSPASLSMAIGEKVTIRCITSTDIDDDMNWYQQKPGEPPKLLISEGNTLRPGVPSRFSSSGYGTDFVFTIENMLSEDVADYYCLQSDNLPYTFGGGTKLEIKR

SEQ ID NO: 11 - protein, 108 aa, Mus musculus
DIELTQSPASLSMPIGEKVTIRCITSTDIDDDMNWYQQKPGEPPKLLISEGNTLRPGVPSRFSSSGYGTDFVFTIENMLSEDVADYYCLQSDNLPYTFGGGTKLEIKR

SEQ ID NO: 12 - protein, 108 aa, Mus musculus
DIELTQSPASLSVATGEKVTIRCITSTDIDDDMNWYQQKPGEPPKLLISEGNTLRPGVPSRFSSSGYGTDFVFTIENMLSEDVADYYCLQSDNLPYTFGGGTKLELKR

SEQ ID NO: 13 - protein, 108 aa, Mus musculus
DIELTQSPASLSMAIGEKVTIRCITSTDIDDDMNWYQQKPGEPPKLLISEGNTLRPGVPSRFSSSGYGTDFVFTIENMLLEDVADYYCLQSDNLPYTFGGGTKLEIKR

SEQ ID NO: 14 - protein, 112 aa, Mus musculus
DIELTQSPASLAVSLGQRATISCRASESVDSYGNSFMHWYQQKPGQPPKLLIYRASNLESGIPARFSGSGSRTDFTLTINPVEADDVATYYCQQSNEDPLTFGAGTKLELKR

SEQ ID NO: 15 - protein, 109 aa, Mus musculus
DIELTQSPAIMSASLGERVTMTCTASSSVSSSYLHWYQQKPGSSPKLWIYSTSNLASGVPARFSGSGSGTSYSLTISSMEAEDAATYYCHQYHRSPFTFGSGTKLEIKR

SEQ ID NO: 16 - protein, 109 aa, Mus musculus
DIELTQSPAIMAASLGQKVTMTCTASSSVSSSYLHWYQQKPGSSPKLWIYSTSNLASGVPARFSGSGSGTSYSLTISSMEAEDAATYYCHQYHRSPFTFGSGTKLEIKR

SEQ ID NO: 17 - protein, 10 aa, Mus musculus
GYTFTNYVIH

SEQ ID NO: 18 - protein, 17 aa, Mus musculus
YINPYNDGTKYNEKFKG

SEQ ID NO: 19 - protein, 12 aa, Mus musculus
FYYGSSIYAVDY

SEQ ID NO: 20 - protein, 10 aa, Mus musculus
GYTFTSYVMH

SEQ ID NO: 21 - protein, 17 aa, Mus musculus
YINPYNDGTKYNEKFKG

SEQ ID NO: 22 - protein, 12 aa, Mus musculus
FYYGSSIYAMDN

SEQ ID NO: 23 - protein, 10 aa, Mus musculus
GYIFTSYVMH

SEQ ID NO: 24 - protein, 17 aa, Mus musculus
YINPYNDVAKYNEKFKG

SEQ ID NO: 25 - protein, 12 aa, Mus musculus
FYYGSSIYAMDY

SEQ ID NO: 26 - protein, 10 aa, Mus musculus
GYTFTSYVMH

SEQ ID NO: 27 - protein, 17 aa, Mus musculus
YINPYNDGTKYNEKFKG

SEQ ID NO: 28 - protein, 12 aa, Mus musculus
FYFGNSVYAVDY

SEQ ID NO: 29 - protein, 10 aa, Mus musculus
GYTFTSYVMH

SEQ ID NO: 30 - protein, 17 aa, Mus musculus
YINPYNDGTKYNEKFKG

SEQ ID NO: 31 - protein, 12 aa, Mus musculus
FYFGNSVYAVDY

SEQ ID NO: 32 - protein, 10 aa, Mus musculus
GFTFTSYWIH

SEQ ID NO: 33 - protein, 17 aa, Mus musculus
EIDPSNGRTNYNEKFKS

SEQ ID NO: 34 - protein, 13 aa, Mus musculus
EGYGYDGYYAMDY

SEQ ID NO: 35 - protein, 10 aa, Mus musculus
GFNIKDTYMH

SEQ ID NO: 36 - protein, 17 aa, Mus musculus
RIDPANGNTKYDPKFQG

SEQ ID NO: 37 - protein, 12 aa, Mus musculus
PHYYGSSYAMDY

SEQ ID NO: 38 - protein, 10 aa, Mus musculus
GFNIKDTYMH

SEQ ID NO: 39 - protein, 17 aa, Mus musculus
RIDPANGNTKYDPKFQG

SEQ ID NO: 40 - protein, 12 aa, Mus musculus
VYYYGSSYAMDY

SEQ ID NO: 41 - protein, 11 aa, Mus musculus
ITSTDIDDAMN

SEQ ID NO: 42 - protein, 7 aa, Mus musculus
EGNTLRL

SEQ ID NO: 43 - protein, 9 aa, Mus musculus
LQSDNLPYT

SEQ ID NO: 44 - protein, 11 aa, Mus musculus
ITSTDIDDDMN

SEQ ID NO: 45 - protein, 7 aa, Mus musculus
EGNTLRP

SEQ ID NO: 46 - protein, 9 aa, Mus musculus
LQSDNLPYT

SEQ ID NO: 47 - protein, 11 aa, Mus musculus
ITSTDIDDDMN

SEQ ID NO: 48 - protein, 7 aa, Mus musculus
EGNTLRP

SEQ ID NO: 49 - protein, 9 aa, Mus musculus
LQSDNLPYT

SEQ ID NO: 50 - protein, 11 aa, Mus musculus
ITSTDIDDDMN

SEQ ID NO: 51 - protein, 7 aa, Mus musculus
EGNTLRP

SEQ ID NO: 52 - protein, 8 aa, Mus musculus
LQSDNLPY

SEQ ID NO: 53 - protein, 11 aa, Mus musculus
ITSTDIDDDMN

SEQ ID NO: 54 - protein, 7 aa, Mus musculus
EGNTLRP

SEQ ID NO: 55 - protein, 9 aa, Mus musculus
LQSDNLPYT

SEQ ID NO: 56 - protein, 15 aa, Mus musculus
RASESVDSYGNSFMH

SEQ ID NO: 57 - protein, 7 aa, Mus musculus
RASNLES

SEQ ID NO: 58 - protein, 9 aa, Mus musculus
QQSNEDPLT

SEQ ID NO: 59 - protein, 12 aa, Mus musculus
TASSSVSSSYLH

SEQ ID NO: 60 - protein, 7 aa, Mus musculus
STSNLAS

SEQ ID NO: 61 - protein, 9 aa, Mus musculus
HQYHRSPFT

SEQ ID NO: 62 - protein, 12 aa, Mus musculus
TASSSVSSSYLH

SEQ ID NO: 63 - protein, 7 aa, Mus musculus
STSNLAS

SEQ ID NO: 64 - protein, 9 aa, Mus musculus
HQYHRSPFT

SEQ ID NO: 65 - protein, 12 aa, Homo sapiens
KFMDVYQRSYCH

SEQ ID NO: 66 - protein, 7 aa, Homo sapiens
EYIFKPS

SEQ ID NO: 67 - protein, 9 aa, Homo sapiens
CNDEFLECV

SEQ ID NO: 68 - protein, 17 aa, Homo sapiens
QIMRIKPHQGQHIGEMS

SEQ ID NO: 69 - protein, 10 aa, Mus musculus
GYTFTNYGMN

SEQ ID NO: 70 - protein, 17 aa, Mus musculus
WINTYTGEPTYAADFKR

SEQ ID NO: 71 - protein, 14 aa, Mus musculus
YPHYYGSSHWYFDV

SEQ ID NO: 72 - protein, 11 aa, Mus musculus
SASQDISNYLN

SEQ ID NO: 73 - protein, 7 aa, Mus musculus
FTSSLHS

SEQ ID NO: 74 - protein, 9 aa, Mus musculus
QQYSTVPWT